(19) United States
(12) Patent Application Publication
(10) Pub. No.: US 2024/0424030 A1
(43) Pub. Date: Dec. 26, 2024

(54) COMPOSITIONS, SYSTEMS, AND METHODS RELATED TO TRANSGENIC HOOKWORMS AS A DRUG PRODUCTION AND DELIVERY TECHNOLOGY

(71) Applicant: Washington University, St. Louis, MO (US)

(72) Inventors: Makedonka Mitreva, St. Louis, MO (US); Sergej Djuranovic, St. Louis, MO (US)

(73) Assignee: Washington University, St. Louis, MO (US)

(21) Appl. No.: 18/755,208

(22) Filed: Jun. 26, 2024

Related U.S. Application Data
(60) Provisional application No. 63/510,315, filed on Jun. 26, 2023.

(51) Int. Cl. A61K 35/62; A01K 67/033; A61K 48/00

(52) U.S. Cl. CPC A61K 35/62; A01K 67/0336; A61K 48/0066; A61K 48/0075; A01K 2217/072; A01K 2227/703

(57) ABSTRACT

Among the various aspects of the present disclosure is the provision of transgenic hookworm compositions and systems and methods of use thereof. The present teachings include compositions for a transgenic hookworm that treats a disease, condition, or indication. Also disclosed is a personal protective biosystem based on the transgenic hookworm, and methods to treat a disease, condition, or indication of a subject using transgenic hookworm compositions.

### CROSS-REFERENCE TO RELATED APPLICATIONS

This application claims the benefit of priority to U.S. Provisional Application Ser. No. 63/510,315 filed on Jun. 26, 2023, which is incorporated herein by reference in its entirety.

### STATEMENT REGARDING FEDERALLY SPONSORED RESEARCH OR DEVELOPMENT

This invention was made with government support under SC1936107 awarded by the Defense Advanced Research Projects Agency (DARPA). The government has certain rights in the invention.

### MATERIAL INCORPORATED-BY-REFERENCE

The Sequence Listing, which is a part of the present disclosure, includes a computer-readable form comprising nucleotide and/or amino acid sequences of the present invention (020608-US-NP_sequences.xml created on 25 Jun. 2024; 11528 bytes). The subject matter of the Sequence Listing is incorporated herein by reference in its entirety.

### FIELD OF THE INVENTION

The present disclosure generally relates to compositions, systems, and methods of use related to drug production and delivery technologies using transgenic hookworms.

### BACKGROUND OF THE INVENTION

Transgenesis as a tractable method has been reported only in a few parasitic nematode species. Although transgenesis is widely used in Caenorhabditis elegans, RNA interference and CRISPR/Cas9 methods have been tested in limited numbers of parasitic nematodes with mixed success. There is an ongoing need for the development of new nematode model systems and associated tools for their molecular biology and genetic engineering.

Until now, hookworms have not been used as a delivery mechanism for other therapeutics, have not produced preventative countermeasures (e.g., against VX for warfighters), and have not been used as locally acting therapeutics.

### SUMMARY OF THE INVENTION

Among the various aspects of the present disclosure is the provision of transgenic hookworm compositions and systems and methods of use thereof.

Briefly, therefore, the present disclosure is directed to a transgenic hookworm technology that can be used as an in vivo drug production and delivery system for a variety of diseases, conditions, and indications.

In one aspect, a composition for the treatment of a disease, condition, or indication in a patient in need, the composition comprising a transgenic hookworm configured to release at least one therapeutic agent, the transgenic hookworm comprising a hookworm genome with a configurable chassis inserted, the configurable chassis comprising: at least one transgenic DNA sequence encoding at least one therapeutic agent, wherein the at least one transgenic DNA sequence encodes the at least one therapeutic agent; at least one promoter DNA sequence operatively linked to the at least one transgenic DNA sequence, the at least one promoter DNA sequence encoding at least one promotor configured to drive expression of the at least one transgenic DNA sequence; and at least one regulatory DNA sequence operatively linked to the at least one transgenic DNA sequence, the at least one regulatory DNA sequence configured to enhance the production of the at least one therapeutic agent. In some aspects, the transgenic hookworm is generated by a transgenesis process selected from adult stage random integration transgenesis, egg stage random integration transgenesis, and targeted transgenesis. In some aspects, the at least one promoter is selected from UBI Schisto, EFT3, ACT2, or UBI. In some aspects, the at least one promoter is EFT3. In some aspects, the at least one regulatory DNA sequence comprises at least one signaling peptide DNA sequence encoding at least one signaling peptide selected from Asp2, Asp1, Ap1, 225, and 382, wherein the at least one signaling peptide is configured to promote translocation of the at least one therapeutic agent to a cell membrane of the transgenic hookworm. In some aspects, the at least one signaling peptide is ASP1. In some aspects, the disease, condition, or indication treated by the composition is selected from exposure to a chemical/biological (CB) agent, an inflammatory/autoimmune disease, or a bacterial/viral infection. In some aspects, the transgenic hookworm comprises a species selected from Ancylostoma caninum or Ancylostoma ceylanicum.

In another aspect, a method to treat a disease, condition, or indication in a subject, the method comprising: providing a composition comprising a transgenic hookworm configured to release at least one therapeutic agent, the transgenic hookworm comprising a hookworm genome with a configurable chassis inserted. The configurable chassis includes at least one transgenic DNA sequence encoding at least one therapeutic agent, wherein the at least one transgenic DNA sequence encodes the at least one therapeutic agent; at least one promoter DNA sequence operatively linked to the at least one transgenic DNA sequence, the at least one promoter DNA sequence encoding at least one promotor configured to drive expression of the at least one transgenic DNA sequence; and at least one regulatory DNA sequence operatively linked to the at least one transgenic DNA sequence, the at least one regulatory DNA sequence configured to enhance production of the at least one therapeutic agent. The method further includes administering the transgenic hookworm to the gut of the subject. In some aspects, the at least one promoter is selected from UBI Schisto, EFT3, ACT2, or UBI. In some aspects, the at least one promoter is EFT3. In some aspects, the at least one regulatory DNA sequence comprises at least one signaling peptide DNA sequence encoding at least one signaling peptide selected from Asp2, Asp1, Ap1, 225, and 382, wherein the at least one signaling peptide is configured to promote translocation of the at least one therapeutic agent to a cell membrane of the transgenic hookworm. In some aspects, the at least one signaling peptide is ASP1. In some aspects, the disease, condition or indication treated is exposure to a chemical/biological (CB) agent, an inflammatory/autoimmune disease, or a bacterial/viral infection. In some aspects, the transgenic hookworm is administered by oral administration of transgenic hookworm ova, transgenic hookworm larvae, transgenic hookworm adult hookworms, and any combination thereof. In some aspects, the transgenic hookworm comprises a species selected from Ancylostoma caninum or Ancylostoma ceylanicum.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 is a schematic of obtaining adult hookworms and transfecting them with an eGFP transgene. Once hookworm eggs develop into the infective larval stage 3 (iL3), they infect a host where they continue to develop. Adult hookworms are isolated from the host and transfected with an eGFP transgene.

FIG. 2A is a set of images of adult hookworms transfected with an eGFP transgene through un-electroporated (left) or electroporated (right) methods.

FIG. 2B is an image of a western showing GFP expression in signal peptides (Asp2, Asp1, Ap1, 225, 382) from A. caninum (ACAN) or A. ceylanicum (ACEY) hookworms.

FIG. 2C is a graph of GFP expression in signal peptides (Asp2, Asp1, Ap1, 225, 382) from A. caninum (ACAN) or A. ceylanicum (ACEY) hookworms.

FIG. 3A is a set of immunofluorescent images of hookworms expressing eGFP (left column), mCherry (middle column), or merged (right column) under the EFT3 promoter (middle row), the SmUbi promoter (bottom row), or an eu-electroporated control (top row).

FIG. 3B is a graph quantifying the GFP and mChery expression under different promoters (UBI Schisto, EFT3, ACT2, UBI) when transfected into hookworms.

FIG. 4A is a set of representative images of GFP expression from hookworm eggs transfected with plasmids under different electroporation conditions. Freshly laid eggs (24 hrs) from adult female hookworms were electroporated without linearized plasmids (grey) or with BchE-EGFP linearized plasmids (green) encoding the GFP reporter gene. Various electroporation voltages and times were tested and transgenesis was evaluated at 96 hours.

FIG. 4B is a set of magnified images from FIG. 4A depicting untransfected (top) and transfected with eGFP (bottom) hookworm eggs which underwent electroporation at 500V for 10 ms.

FIG. 4C is a graph of detected fluorescence of GFP from eGFP-transfected hookworm eggs from FIG. 4A. Hookworm eggs were transfected with and without electroporation at 300V, 400V, and 500V for 5 ms, 10 ms, or 20 ms.

FIG. 4D is a sect of graphs of ddPCR (left) and fluorescence (right) of untreated and GFP-transfected hookworm eggs.

FIG. 5A is a schematic of the identification at GSH for targeted integration using GRISPR/Cas9-mediated knock-in or reporter genes in adult and egg stages of A. ceylanicum.

FIG. 5B is an image of agarose gel electrophoresis showing expected PCR products that indicate the integration of the RFP reporter gene in the adult hookworm genome.

FIG. 5C is a set of representative images (top) showing the expression of the RFP reporter (red) and bright field (white) in hookworm eggs. The table (bottom) depicts the electroporation settings, including the number of pulses, the pulse width (duration), and the voltage.

FIG. 5D is an image of Sanger sequencing which confirms the integration of the RFP reporter gene into the hookworm genome using M13 (top) and T7 (bottom) primers.

FIG. 6 is a schematic depicting the use of hookworms as a drug production and delivery technology. Adult hookworms are modified using CRISPR to express molecules to neutralize or treat a disease, toxin, or infection.

FIG. 7A is a schematic of the double-strand donor construct and the genome safe harbor (GSH) region, identified in the genomic sequence upstream of prioritized target gene maker-ANCCEYDFT_Contig13-pred_gff_snap-gene-4.9. The genetic components of the double-strand donor construct and its insertion into the A. ceylanicum genome at the GSH region of the “ANCCEYDFT_contig13” contig. The construct was engineered by cloning the human single-chain antibody fragment (S16-HuScFv) previously selected from a human ScFv phage display library and shown to neutralize tetrodotoxin (Chulanetra et al, 2012) into pCDNA3.1(+) backbone (at Notl restriction site), with CMV promoter and BGH terminator. The donor construct was flanked at its termini with approx. 600 bp HA.

FIG. 7B is a schematic of the region between the left homology arm (LHA, SEQ ID NO:8) and the right homology arm (RHA, SEQ ID NO:10) from FIG. 7A. The LHA is situated at the position of two overlapping sgRNAs, sgRNA 1 (SEQ ID NO:6, ranked 5th by CHOPCHOP) and sgRNA 2 (SEQ ID NO:7, ranked 13th). Both sgRNA sequences end with a CGG protospacer adjacent motif.

FIG. 7C is a graph of the average gene expression level of maker-ANCCEYDFT_Contig13-pred_gff_snap-gene-4.9 (log-scale FPKM) across the A. ceylanicum life cycle. The average expression level of all genes across all stages is indicated by the dashed red line.

FIG. 7D is a graph of the gene expression level of genes across the GSH region. X-axis positions indicate gene coordinates and Y-axis values represent gene expression levels. The prioritized target gene maker-ANCCEYDFT_Contig13-pred_gff_snap-gene-4.9 is indicated in blue, and the average expression level of all genes across all stages is indicated by the dashed red line. The GSH region is indicated by a purple-shaded area running vertically up the plot. There is 12,966 bp of sequence preceding the target gene on the genome assembly.

FIG. 8 is a graph of in vitro neutralization of TTX by recombinant S16-HuScFv antibody fragments in the presence of media (EMEM) or hookworm ES products. Data presented as mean±standard deviation (SD); n=5. *, indicates p<0.05. The purpose of this study was to demonstrate the ability of the recombinant CMs to neutralize TTX. The neutralization ability of CMs was assessed using recombinant S16-HuScFv was incubated with 200 nM TTX before in vitro exposure to Neura-2a cells. The cells are then exposed to the ion-channel mediators veratridine and ouabain are monitored for osmotic lysis using the Promega CellTox Green Cytotoxicity Assay for up to 72 hours. Any TTX that is not neutralized via CM will negate the effect of the veratridine and ouabain, resulting in increased viability of cells.

FIG. 9A is a schematic of transgene produced and secreted by adult hookworm using programmed CRISPR-Cas9 KI of a donor construct using electroporation. Targeted knock-in of the transgene is performed on F0 adults harvested from the intestine of hamsters (n=3).

FIG. 9B is a graph of transgenes produced and secreted by adult hookworms using programmed CRISPR-Cas9 KI of a donor construct using electroporation. TTX neutralization was assayed incubating samples with 200 nM TTX before in-vitro exposure to Neura-2a cells. TTX inhibition of osmotic lysis of the cells by the ion-channel mediators veratridine (V; 0.2 mM) and ouabain (O; 0.4 mM) is monitored using the Promega CellTox Green Cytotoxicity Assay for up to 72 hours. TTX neutralization is quantified between 0% (V+O+TTX, with no countermeasure) and 100% (V+O, with no TTX) based on relative fluorescence units, adjusted for dilution factors. Error bars represent the standard deviation based on three technical replicates. P values represent one-sided T-tests compared to the V+O+TTX control neutralization values.

FIG. 10A is a set of images of the egg stage of the human hookworm A. ceylanicum which was targeted for germ-line transfection with the donor transgene. Immature eggs extracted from the intestine of a hamster at 20 days post-infection (dpi) were observed at day 0 in BU buffer.

FIG. 10B is a set of images of the egg stage of the human hookworm A. ceylanicum which was targeted for germ-line transfection with the donor transgene. Immature eggs extracted from the intestine of a hamster at 20 days post-infection (dpi) were observed at day 1 in BU buffer.

FIG. 10C is a set of images of mature hookworm eggs extracted from hamster fecal samples at 20 dpi and observed at day 0 in PBS+ABX.

FIG. 10D is an image of mature hookworm eggs extracted from hamster fecal samples at 20 dpi and observed on day 1 in PBS+ABX.

FIG. 11A is a schematic of the targeted knock-in of S16-HuScFv in hookworm eggs and evidence for germ-line transgenesis using the CRISPR-Cas system and a hamster model for human hookworm A. ceylanicum infection.

FIG. 11B is a set of images of gel electrophoresis showing targeted knock-in of the transgene S16-HuScFv into F0 eggs. Expected amplicon sizes of 2,365 bp and 1,061 bp. Controls included electroporation with no donor DNA or RNP complex, and electroporation with donor DNA but no RNP complex. The integrity of the DNA was confirmed by PCR amplification of the Rab 11a/b constitutive gene (“Control PCR”, expected amplicon size 1,000 bp).

FIG. 11C is a set of images of gel electrophoresis confirming the targeted knock-in of the transgene S16-HuScFv in F0 adults harvested from the intestine of hamsters (n=3) by genomic DNA PCR, using 5′up F3 and CMV_R primers (expected amplicon size 1,061 bp). The integrity of the DNA was confirmed by the PCR amplification of the Rab 11a/b constitutive gene (“Control PCR”, expected amplicon size 1,000 bp).

FIG. 11D is a set of images of gel electrophoresis confirming targeted knock-in of the transgene S16-HuScFv in F1 eggs from 3 hamster hosts by PCR (eggs collected on days 18, 19 and 20 of infection), using 5′up F3 and CMV R primers (expected amplicon size of 1,061 bp). The integrity of the DNA was confirmed by the PCR amplification of the FPP constitutive gene (“Control PCR”, expected amplicon size of 350 bp).

FIG. 12 is a schematic of the targeted insertion of S16-HuScFv into the genome of the egg stage of the human hookworm Ancylostoma ceylanicum. Knock-in verification by sequencing of the targeted genomic region using Oxford Nanopore Technology. ONT sequencing was performed on F0 transgenic eggs (pool of eggs, dark green; 5′up F3 and S16 R primers), F0 adults (single worms, blue; 5′up F3 and CMV R primers), and F1 transgenic eggs (pool of eggs, light green; 5′up F3 and CMV R primers). The transgenic construct is shown in the A. ceylanicum genome. All Oxford Nanopore sequences exactly matched the expected transgenic genomic sequences.

FIG. 13A is a schematic of a programmed CRISPR-Cas knock-in experiment using a hamster model for human hookworm A. ceylanicum infection to neutralize tetrodotoxin (TTX).

FIG. 13B is a graph of secreted S16-HuScFv by transgenic F0 adults detected by western blot using an anti-Kappa antibody, expected band size 30 kDa. Neutralization of TTX by serum samples taken from hamsters infected with WT and S16 TG iL3 A. ceylanicum. TTX neutralization was assayed incubating samples with 200 nM TTX before in-vitro exposure to Neura-2a cells. TTX inhibition of osmotic lysis of the cells by the ion-channel mediators veratridine (V; 0.2 mM) and ouabain (O; 0.4 mM) is monitored using the Promega CellTox Green Cytotoxicity Assay for up to 72 hours. TTX neutralization is quantified between 0% (V+O+TTX, with no countermeasure) and 100% (V+O, with no TTX) based on relative fluorescence units, adjusted for dilution factors. Error bars represent the standard deviation based on three technical replicates for the technical controls, LNP, LNP+elec, and the standard deviation for biological replicates for the serum samples from hamsters infected with WT A. ceylanicum (N=2) and electroporated transgenic A. ceylanicum expressing S16 (N=4; marked with asterisks). P values represent one-sided T-tests compared to the V+O+TTX control neutralization values.

FIG. 14 is a schematic of the bioinformatic selection of an optimal GSH region. Differential 5′ upstream sequence analysis was performed to identify sequence motifs associated with genes with high gene expression across the A. ceylanicum life cycle. This prioritizes sequences and genomic regions with the highest probability of being constantly accessible, regardless of stage. Then, to narrow down to a single genomic region, a single A. ceylanicum gene was prioritized. With the upstream sequence motif and high gene expression across the life cycle, a single peptide, and proteomic evidence for secretion increased the likelihood of a GSH region. Finally, the GSH region was refined, and a specific CRISPR target site was identified.

FIG. 15A is a table of electroporation protocols (top) and the corresponding gel of the PCR product (bottom) to optimize electroporation parameters for target transgenesis in A. ceylanicum eggs. In vitro, laid eggs were electroporated using 24 pre-programmed protocols with varying parameters for pulse voltage, width, and number. Confirmation of electroporation was performed by PCR amplification of a transgene-specific forward primer and a downstream genomic reverse primer (expected amplicon size of 350 bp).

FIG. 15B is a table (top) and corresponding gel (bottom) of electroporation parameters. The best results from FIG. 15A, lanes 12-19, were repeated to optimize the electroporation parameters.

FIG. 15C is a table (top) and corresponding gel (bottom) of electroporation parameters. The best results from FIG. 15B, lanes 12 and 13, were repeated to optimize the electroporation parameters.

FIG. 16 is a graph of the in vitro response of CellTox Green assay fluorescence for anti-TTX antibody fragments in hookworms with the transgenic S16-HuScFv gene (V-veratridine; O-ouabain; TTX-tetrodotoxin).

FIG. 17A is an image of hookworms in the human duodenum (left) and the production of hookworm peptides (green) and prevent bacteria (blue) from interacting with human cells and causing an immune response.

FIG. 17B is a schematic of the configurable chassis, which can have one or more promoters driving one or more transgenes, connected by a linker.

### DETAILED DESCRIPTION OF THE INVENTION

The present disclosure is based, at least in part, on the discovery that hookworms can be genetically modified to produce a drug production and delivery system. As described in the examples herein, multiple approaches to create transgenic hookworms were developed, and the use of transgenic hookworms in personal protective biosystems is characterized.

One aspect of the present disclosure provides for compositions, systems, and methods related to hookworm drug production and delivery technology.

In one aspect, a composition that treats a disease, condition, or indication is disclosed that includes a transgenic hookworm. The transgenic hookworm includes a configurable chassis inserted into the hookworm genome to facilitate the insertion of at least one gene encoding at least one therapeutic agent under the control of at least one distinct promoter and at least one regulatory element. The transgenic hookworm further includes at least one gene inserted into the chassis, wherein expression of the at least one gene releases the at least one therapeutic agent for a disease, condition, or indication. The transgenic hookworm further includes at least one promoter gene inserted into the chassis. The transgenic hookworm further includes at least one regulatory element inserted into the chassis. In some aspects, the transgenic hookworm is generated by a transgenesis method selected from adult stage random integration transgenesis, egg stage random integration transgenesis, and targeted transgenesis. In some aspects, the at least one promoter gene is EFT3. In some aspects, the at least one regulatory element comprises a gene encoding a signaling peptide, wherein expression of the signaling peptide increases the production of the therapeutic agent. In some embodiments, the signaling peptide is ASP1. In some aspects, the disease, condition or indication treated by the composition treats comprises exposure to a chemical/biological (CB) agent. In other embodiments, the disease, condition, or indication treated by the composition comprises an inflammatory/autoimmune disease.

In another aspect, a personal protective biosystem is disclosed that includes a transgenic hookworm. The transgenic hookworm includes a configurable chassis inserted into the hookworm genome to facilitate the insertion of at least one gene encoding at least one therapeutic agent under the control of at least one distinct promoter and at least one regulatory element. The transgenic hookworm further includes at least one gene inserted into the chassis, wherein expression of the at least one gene releases the at least one therapeutic agent for a disease, condition, or indication. The transgenic hookworm further includes at least one promoter gene inserted into the chassis. The transgenic hookworm further includes at least one regulatory element inserted into the chassis. In some aspects, the transgenic hookworm is generated by a transgenesis method selected from adult stage random integration transgenesis, egg stage random integration transgenesis, and targeted transgenesis. In some aspects, the at least one promoter gene is EFT3. In some aspects, the at least one regulatory element comprises a gene encoding a signaling peptide, wherein expression of the signaling peptide increases the production of the therapeutic agent. In some embodiments, the signaling peptide is ASP1. In some aspects, the disease, condition or indication treated by the composition treats comprises exposure to a chemical/biological (CB) agent. In other embodiments, the disease, condition, or indication treated by the composition comprises an inflammatory/autoimmune disease.

In an additional aspect, a method to treat a disease, condition, or indication in a subject is disclosed that includes providing a composition comprising a genetically modified hookworm and administering the genetically modified hookworm to the gut of the subject. In some aspects, providing the composition comprising a genetically modified hookworm further comprises genetically modifying a hookworm by inserting a configurable chassis into a genome of the hookworm to facilitate the insertion of at least one gene encoding at least one therapeutic agent under the control of at least one distinct promoter and at least one regulatory element, inserting the at least one gene into the chassis, wherein expression of the at least one gene releases the at least one therapeutic agent for a disease, condition, or indication, inserting at least one promoter gene into the chassis, and inserting at least one regulatory element into the chassis. In one aspect, the genetically modified hookworm is generated by a transgenesis method selected from adult stage random integration transgenesis, egg stage random integration transgenesis, and targeted transgenesis. In some embodiments, the at least one promoter gene is EFT3. In some embodiments, the at least one regulatory element comprises a gene encoding a signaling peptide, wherein expression of the signaling peptide increases the production of the therapeutic agent. In some embodiments, the signaling peptide is ASP1. In some embodiments, the disease, condition or indication to be treated by the method can be exposure to a chemical/biological (CB) agent. In other embodiments, the disease, condition or indication to be treated by the method can be an inflammatory/autoimmune disease.

In accordance with an aspect of the present disclosure, different transgenesis (TG) approaches to knock in a transgene in the adult and egg stage of the hookworm Ancylostoma ceylanicum are evaluated.

In some embodiments, electroporation of adult stages of A. ceylanicum hookworms with pre-linearized plasmid DNAs encoding the GFP reporter gene is performed. Random integration can be assayed for DNA insertion with fluorescence microscopy and expression of tagged reporter proteins, and random integration TG was confirmed. In some embodiments, 5 different signal peptides for secretions and b) 4 different promoters were evaluated to identify one that enables strong GFP expression. In an exemplary embodiment, the best-performing signal peptide was ASP1 and promoter EFT3, which had 3.8-fold more GFP production compared to WT adult worms.

In some embodiments, freshly laid eggs (24 hrs) from adult female hookworms were electroporated with pre-linearized plasmid DNAs, encoding the GFP reporter gene, and evaluated for transgenesis at 96 hrs. GFP+ve eggs indicated egg TG and ddPCR values indicated GFP transcript expression.

In some embodiments, targeted transgenesis of hookworms can be performed. In some embodiments, CRISPR/Cas9-mediated knock-in of reporter genes, in adult and egg stages of A. ceylanicum, can be used for targeted transgenesis. In some embodiments, Cas9:gRNA complexes and genome-safe harbors can be used for the estimation of Cas9 efficacy and knock-in. GSH can be evaluated based on Multi-omics helminth databases. In an exemplary embodiment, the electroporation and Neon electroporation systems provided superior transgenesis for A. ceylanicum eggs. All 24 neon protocols were evaluated as protocols for knock-in (KI). Evaluation of collected samples for transgenesis can be done by PCR, Sanger and NGS sequencing of amplicons, as well as western blot analyses for expressed reporters.

The present disclosure indicates overall successful transgenesis of A. ceylanicum worms with the use of different and stage specific methods. In a broad embodiment, A. ceylanicum can be a novel and tractable model parasitic nematode system that can be used for genetic engineering.

In one aspect of the present disclosure, the transgenic hookworm system can be used as a personal protective biosystem. In another aspect, the hookworm system can be a drug production and delivery system. In accordance with another aspect, the system can have prophylactic and therapeutic applications. In another aspect, the system can provide long-lasting production and targeted delivery. In another aspect, the system can protect a warfighter from chemical and biological (CB) agents with pre-exposure CB protection by a released therapeutic agent. In an exemplary embodiment, the system can provide protection to warfighters against organophosphates or toxins.

In some aspects, diseases, disorders, and indications can benefit from the long-lasting delivery of therapeutics to the gastrointestinal tract that the hookworm system can provide. In some embodiments, these diseases, disorders, and disorders can include gut inflammatory/autoimmune diseases (IBD; Crohn's disease, ulcerative colitis, celiac disease, esophagitis) and multidrug-resistant bacterial infections to be treated through secretion of antibacterial peptides. In accordance with another aspect, the technology can be used for a range of diseases, disorders, and indications, including but not limited to inflammatory diseases, bacterial infections, and CB exposure. In one aspect, the transgenic hookworm is engineered for slow drug release of beneficial compounds.

In one aspect, the technology takes advantage of the sophisticated secretory system of this eukaryotic organism. In another aspect, the technology can have the ability to constantly produce therapeutic molecules. In another aspect, the technology can take advantage of a genetically altered form of a hookworm via a configurable chassis. In yet another aspect, the configurable chassis can be modified to enable the rapid exchange of therapeutic genes and optimized for high levels of production and secretion. In some aspects, the genetically altered hookworm can be modified with a configurable chassis to enable rapid exchange of various therapeutic genes, including but not limited to encoding enzymes, peptides, and antibodies. In some aspects, the technology can provide prophylactic and therapeutic value because it can reside in the human gut, can be long-lived in the gut, and can enable continuous release of the biologic and local delivery of therapeutics to the gut.

In various aspects, the use of hookworms as a molecular foundry harnesses the hookworm's sophisticated secretory system and host immune-modulatory functions to significantly improve their ability to generate and deliver bio-actives on demand. A configurable chassis has been designed for hookworm transgenesis to allow the insertion of multiple genes encoding different biologics under the control of distinct promoters and regulatory elements that were systematically and methodically designed and optimized.

Controlled hookworm administration has been shown to be safe and well-tolerated. Wild-type hookworms have been used therapeutically to suppress gut inflammations and elevate disease symptoms via localized immunomodulation. In one aspect, the co-evolution of the hookworm can be safe, tolerable, and flexible.

### Disease Modulation Agents

As described herein, the expression of various factors has been implicated in various diseases, disorders, and conditions. As such, modulation of disease-associated factors (e.g., modulation of inflammatory factors) can be used for treatment of such conditions. A disease modulation agent can modulate a disease response or induce or inhibit a disease. Disease modulation can comprise modulating the expression of disease factors on cells, modulating the quantity of cells that express disease factors, or modulating the quality of the disease-factor expressing cells.

Disease modulation agents can be any composition or method that can modulate disease factor expression on cells (e.g., inflammatory factors). For example, a disease modulation agent can be an activator, an inhibitor, an agonist, or an antagonist. As another example, the disease modulation can be the result of gene editing.

A disease modulation agent can be an antibody (e.g., a monoclonal antibody to a disease factor).

A disease-modulating agent can be an agent that induces or inhibits progenitor cell differentiation into disease-factor expressing cells (e.g., inflammatory cells or bacterial cells). For example, factors produced by the transgenic system can be used to block disease factors.

Disease Signal Reduction, Elimination, or Inhibition by Small Molecule Inhibitors, shRNA, siRNA, or ASOs

As described herein, a disease modulation agent can be used for use in therapy for a variety of diseases. A disease modulation agent can be used to reduce/eliminate or enhance/increase signals associated with the disease. For example, a disease modulation agent can be a small molecule inhibitor of a disease. As another example, a disease modulation agent can be a short hairpin RNA (shRNA). As another example, a disease modulation agent can be a short interfering RNA (siRNA).

As another example, RNA (e.g., long noncoding RNA (lncRNA)) can be targeted with antisense oligonucleotides (ASOs) as a therapeutic. Processes for making ASOs targeted to RNAs are well known; see e.g. Zhou et al. 2016 Methods Mol Biol. 1402:199-213. Except as otherwise noted herein, therefore, the process of the present disclosure can be carried out in accordance with such processes.

### Disease-Inhibiting Agent

One aspect of the present disclosure provides for targeting of a disease factor, its receptor, or its downstream signaling. The present disclosure provides methods of treating or preventing a disease based on the discovery that hookworms can be genetically modified to provide long-term and continuous drug production and delivery.

As described herein, inhibitors of a disease (e.g., antibodies, fusion proteins, small molecules) can reduce or prevent disease. A disease-inhibiting agent can be any agent that can inhibit a disease factor, downregulate a disease factor, or knock down a disease factor.

As an example, a disease-inhibiting agent can inhibit inflammatory signaling.

For example, the disease-inhibiting agent can be an antibody that binds and inhibits a disease factor. Furthermore, the antibody can be a murine antibody, a humanized murine antibody, or a human antibody.

As another example, the disease-inhibiting agent can be an antibody, wherein the antibody prevents binding of the disease factor to its receptor, and prevents activation of the disease factor and downstream signaling.

As another example, the disease-inhibiting agent can be a fusion protein. For example, the fusion protein can be a decoy receptor for a disease factor. Furthermore, the fusion protein can comprise a mouse or human Fc antibody domain fused to the ectodomain of a disease factor.

As another example, a disease-inhibiting agent can be a transgenic hookworm composition, which has been shown to be a potent and specific inhibitor of disease factor signaling.

As another example, a disease-inhibiting agent can be an inhibitory protein that antagonizes a disease factor. For example, the disease-inhibiting agent can be a viral protein, which has been shown to antagonize a disease factor.

As another example, a disease-inhibiting agent can be a short hairpin RNA (shRNA) or a short interfering RNA (siRNA) targeting a disease factor.

As another example, a disease-inhibiting agent can be an sgRNA targeting a disease factor.

Methods for preparing a disease-inhibiting agent (e.g., an agent capable of inhibiting disease factor signaling) can comprise the construction of a protein/Ab scaffold containing the natural disease factor receptor as a neutralizing agent; developing inhibitors of the disease factor receptor “downstream”; or developing inhibitors of the disease factor production “up-stream”.

Inhibiting a disease can be performed by genetically modifying a disease factor in a subject or genetically modifying a subject to reduce or prevent expression of the disease factor gene, such as through the use of CRISPR-Cas9 or analogous technologies, wherein, such modification reduces or prevents a disease.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high-performance liquid chromatography. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or other counterions. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs the transcription of a nucleic acid. An inducible promoter is generally understood as a promoter that mediates the transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position+1. With respect to this site, all other sequences of the gene and its controlling regions can be numbered. Downstream sequences (i.e., further protein-encoding sequences in the 3′ direction) can be denominated positive, while upstream sequences (mostly of the controlling regions in the 5′ direction) are denominated negative.

“Operably linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects the expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid that is replaced has a similar property as the original amino acid, for example, the exchange of Glu by Asp, Gln by Asn, Val by IIe, Leu by IIe, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. The amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in vitro using the specific codon usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41 (fraction G/C content)−0.63(% formamide)−(600/I). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated into the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| | |
|---|---|
| Side Chain Characteristic | Amino Acid |

Non-polar (hydrophobic)

| | |
|---|---|
| A. Aliphatic: | A L I V P |
| B. Aromatic: | F W |
| C. Sulfur-containing: | M |
| D. Borderline: | G |

Uncharged-polar

| | |
|---|---|
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desire to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, disease-associated signals can be modulated (e.g., reduced, eliminated, or enhanced) using genome editing. Processes for genome editing are well known; see e.g. Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of a disease factor by genome editing can result in protection from autoimmune or inflammatory diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for the treatment of a variety of diseases to target cells by the removal of disease-associated signals.

For example, the methods as described herein can comprise a method for altering a target polynucleotide sequence in a cell comprising contacting the polynucleotide sequence with a clustered regularly interspaced short palindromic repeats-associated (Cas) protein.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption-delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce the dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of the agent being metabolized or excreted from the body. The controlled release of an agent may be stimulated by various inducers, e.g., a change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

### Therapeutic Methods

Also provided is a process of treating, preventing, or reversing a disease in a subject in need of administration of a therapeutically effective amount of a transgenic hookworm composition, so as to treat a disease.

Methods described herein are generally performed on a subject in need thereof. A subject in need of the therapeutic methods described herein can be a subject having, diagnosed with, suspected of having, or at risk for developing a disease or condition. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a transgenic hookworm composition is, for example, an amount that would cause the desired therapeutic effect in a subject while minimizing undesired side effects. In various embodiments, an effective amount of a transgenic hookworm composition described herein can substantially inhibit a disease, condition, or indication slow the progress of a disease, condition, or indication, or limit the development of a disease, condition, or indication.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration. In one exemplary aspect, the disclosed transgenic hookworms are administered orally in ova form, larvae form, adult form, and any combination thereof.

In various aspects, any suitable species of hookworm may be modified to produce the transgenic hookworms as described herein including, but not limited to, Ancylostoma caninum or Ancylostoma ceylanicum.

When used in the treatments described herein, a therapeutically effective amount of a transgenic hookworm composition can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat a disease, condition, or indication.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a transgenic hookworm composition can occur as a single event or over a time course of treatment. For example, a transgenic hookworm composition can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accordance with the methods described herein can be performed prior to, concurrent with, or after conventional treatment modalities for a variety of diseases.

A transgenic hookworm composition can be administered simultaneously or sequentially with another agent, such as an antibiotic, an anti-inflammatory, or another agent. For example, a transgenic hookworm composition can be administered simultaneously with another agent, such as an antibiotic or an anti-inflammatory. Simultaneous administration can occur through the administration of separate compositions, each containing one or more of a transgenic hookworm composition, an antibiotic, an anti-inflammatory, or another agent. Simultaneous administration can occur through the administration of one composition containing two or more of a transgenic hookworm composition, an antibiotic, an anti-inflammatory, or another agent. A transgenic hookworm composition can be administered sequentially with an antibiotic, an anti-inflammatory, or another agent. For example, a transgenic hookworm composition can be administered before or after the administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. In one exemplary embodiment, the transgenic hookworm composition disclosed herein is administered orally.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

### Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to a transgenic hookworm composition and factors to facilitate gut delivery. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

The methods and algorithms of the invention may be enclosed in a controller or processor. Furthermore, methods and algorithms of the present invention can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Creation of Transgenic Hookworms

To develop a transgenic hookworm system in accordance with the present disclosure, the following experiments were conducted. This example describes methods to generate a transgenic hookworm system through three distinct methods. Transgenic hookworms were successfully generated through adult stage random integration transgenesis, egg stage random integration transgenesis, and targeted transgenesis.

Transgenesis as a tractable method has been reported only in a few parasitic nematode species while it is widely used in Caenorhabditis elegans. RNA interference and CRISPR/Cas9 methods have been tested in limited numbers of parasitic nematodes however with mixed success. There is an ongoing need for the development of new nematode model systems and associated tools for their molecular biology and genetic engineering. We evaluated different transgenesis (TG) approaches to knock in transgene in the adult and egg stages of the hookworm Ancylostoma ceylanicum.

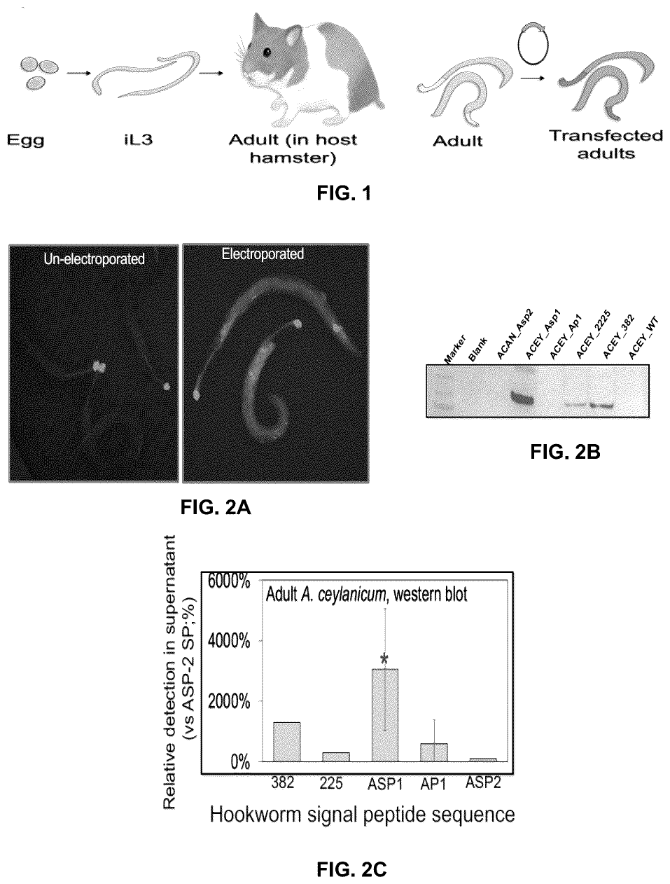

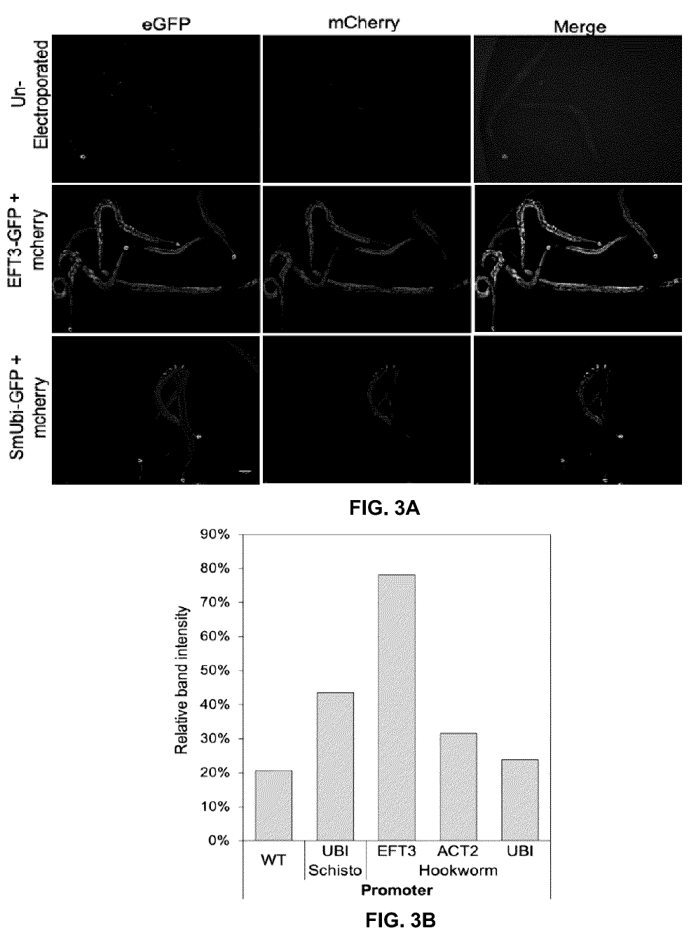

Random integration transgenesis into adult stage hookworms was optimized with an eGFP transgene. A. ceylanicum eggs were grown to infective larval stage 3 (iL3), grown to the adult stage in a host hamster, harvested, and transfected with an eGFP transgene (FIG. 1). Adult stage A. ceylanicum were electroporated with pre-linearized plasmid DNAs encoding a GFP reporter gene. Random integration was assayed for DNA insertion by fluorescence microscopy (FIG. 2A) and expression of tagged reporter proteins (FIG. 2B), while un-electroporated hookworms were unable to take up and integrate the transgene. Once the random integration TG was confirmed we evaluated 5 different signal peptides to promote protein translocation to the cell membrane and 4 promoters to drive gene expression. Based on the differential secretion of the signal peptides (382, 225, Asp1, Ap1, Asp2) the best-performing signal peptide was ASP1 (FIG. 2B, FIG. 2C). The promoter EFT3 had 3.8-fold more GFP production compared to WT adult worms (FIG. 3A, FIG. 3B).

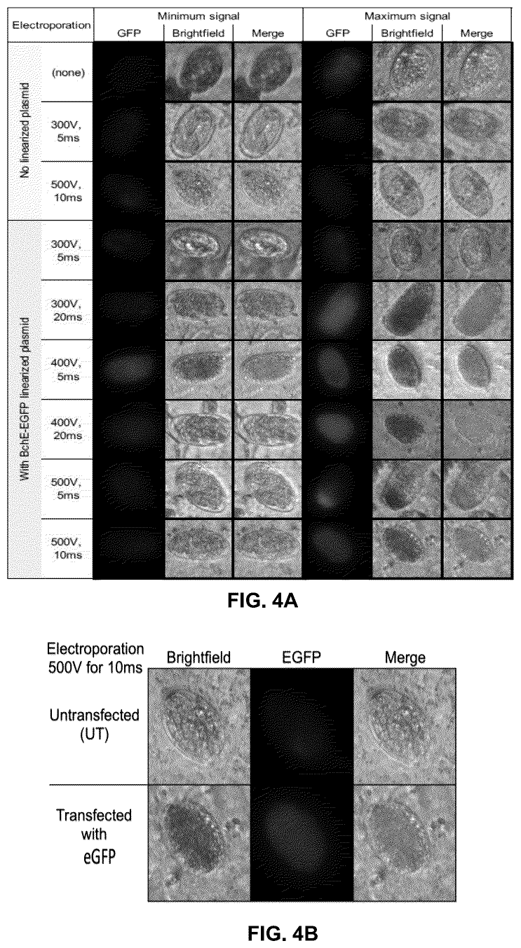

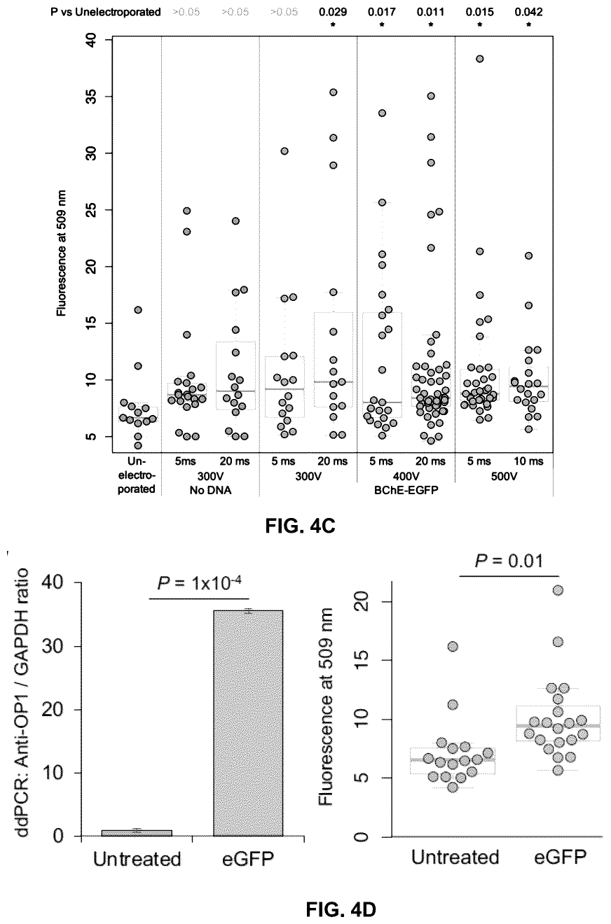

Random integration transgenesis into egg stage hookworms was also evaluated. Freshly laid eggs (24 hrs) from adult female hookworms were electroporated with pre-linearized plasmid DNA encoding the GFP reporter gene and evaluated for transgenesis. Electroporation conditions were optimized by changing the voltage and duration and then assessing GFP expression after 96 hours. Electroporation at 300, 400, and 500 volts for 5, 10, and 20 ms was tested and fluorescence graphed (FIG. 4A, FIG. 4B, FIG. 4C). Additionally, integration of GFP was examined by ddPCR and fluorescence compared to untreated control (FIG. 4D).

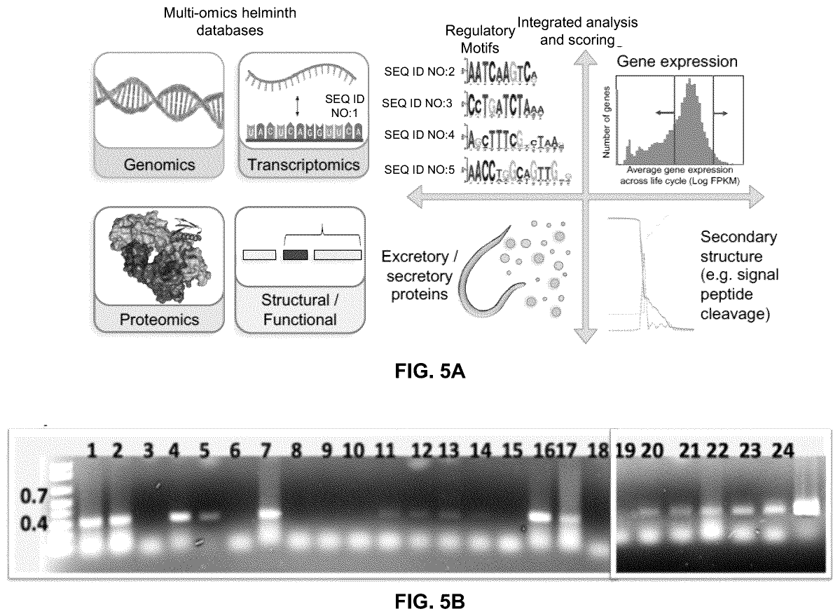

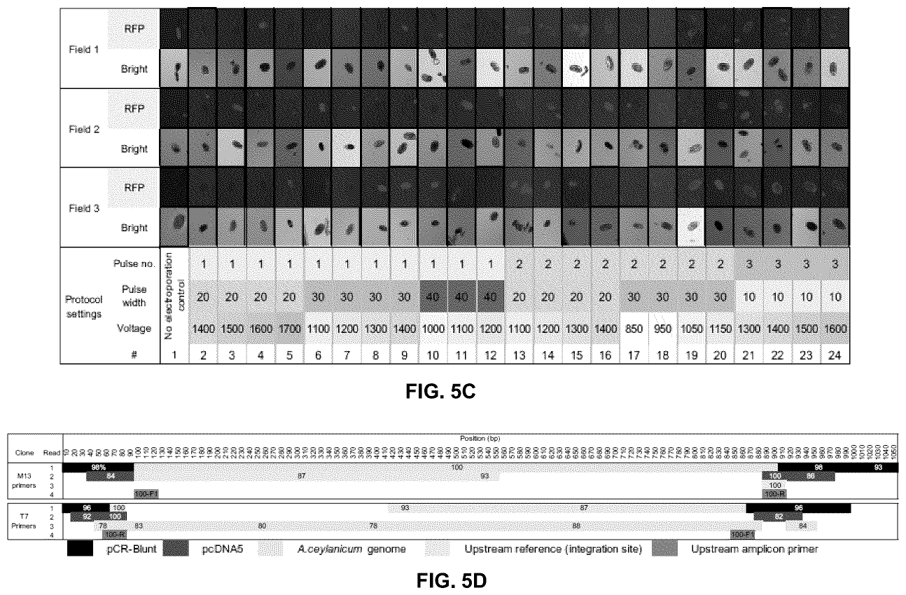

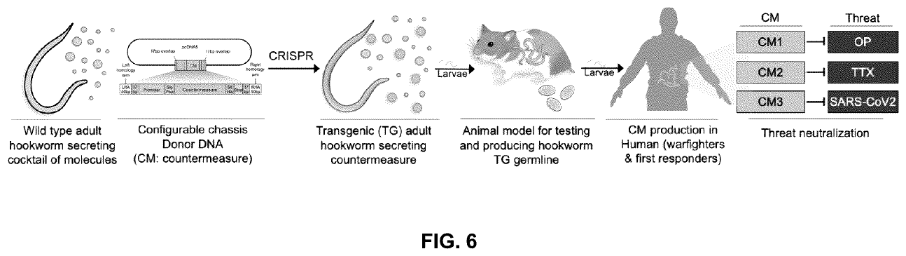

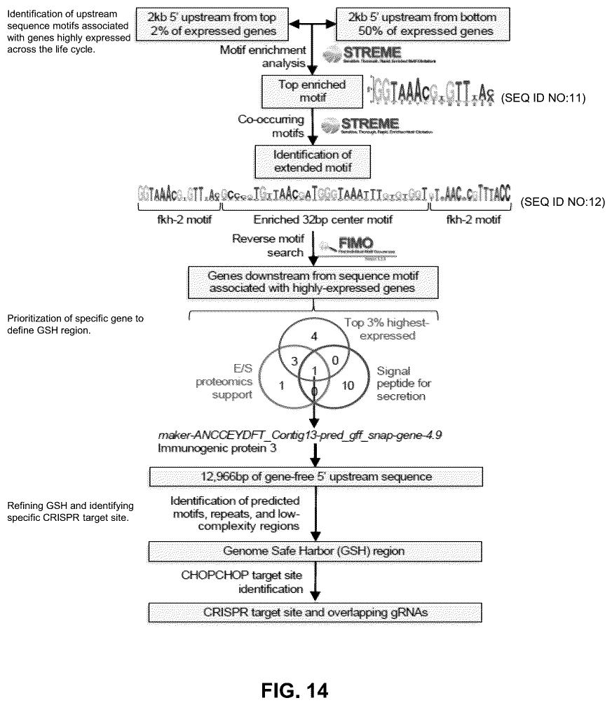

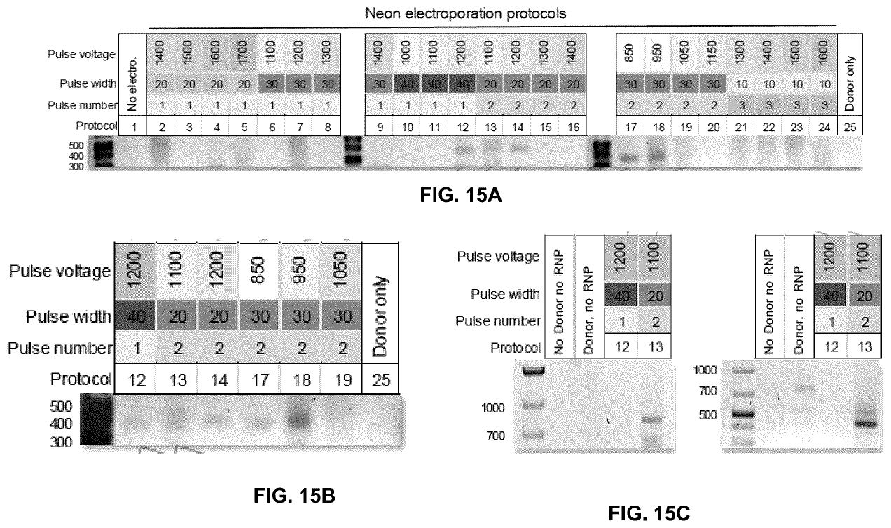

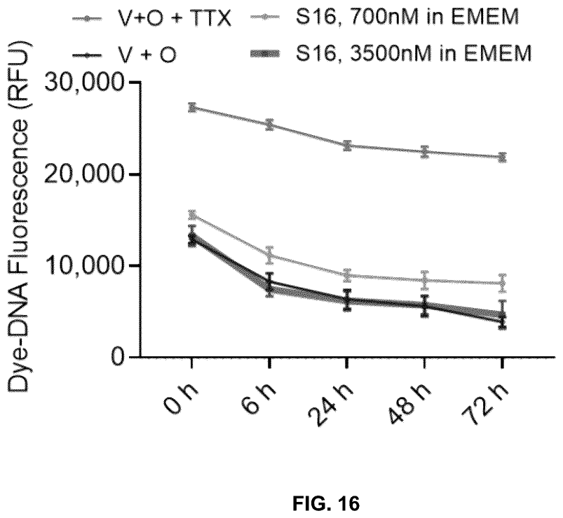

Targeted transgenesis into egg and adult stage hookworms was performed using a CRISPR/Cas-9 mediated knock-in of reporter genes. To estimate Cas9 efficacy and knock-in of the target gene, Cas9, gRNA complexes and genome safe harbors (GSH) were evaluated based on Multi-omics helminth databases. Regulatory motifs (SEQ ID NOS:2, 3, 4, 5, 11, and 12) gene expression, secretory proteins, and secondary structures were evaluated for integrated analysis and scoring of GSH (FIG. 5A, FIG. 14). Integration of RFP into adult hookworms was confirmed by gel electrophoresis (FIG. 5B) and fluorescence confirmed RFP knock-in into hookworm eggs (FIG. 5C, FIG. 15A, FIG. 15B, FIG. 15C). Evaluation of all collected samples for transgenesis was further confirmed by Sanger sequencing (FIG. 5D). Integration of transgenes can be accomplished through random integration or targeted transgenesis at both the egg and adult stages of A. ceylanicum. Through the integration of a configurable chassis, a transgenic adult hookworm can secrete a variety of different proteins including, but not limited to, countermeasures to various biological and chemical threats, as well as therapeutics for inflammatory or autoimmune diseases (FIG. 6).

### Example 2—Personal Protective Biosystems

To characterize the use of the transgenic hookworm system of the present disclosure as a personal protective biosystem for a variety of applications, including protection against chemical/biological (CB) agents, and treatment of inflammatory diseases and bacterial infections, the following experiments were conducted.

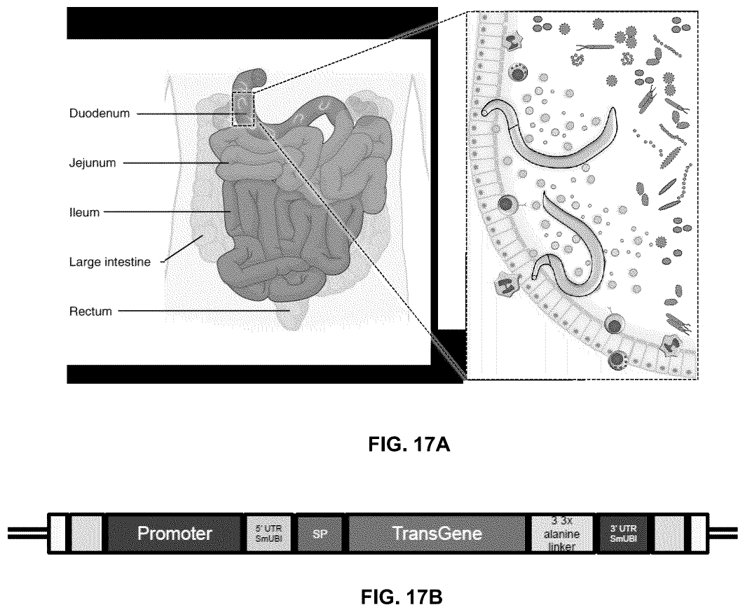

The use of hookworms as a molecular foundry is a novel concept that promises to harness the hookworm's sophisticated secretory system and host immune to modulate functions to significantly improve their ability to generate and deliver bio-actives on demand. With the integration of a configurable chassis, the modified hookworm can enable the rapid exchange of therapeutic genes and be optimized for high levels of production and secretion. Multiple genes encoding different biologics under the control of distinct promoters and regulatory elements can be designed into the configurable chassis (FIG. 6, FIG. 17B).

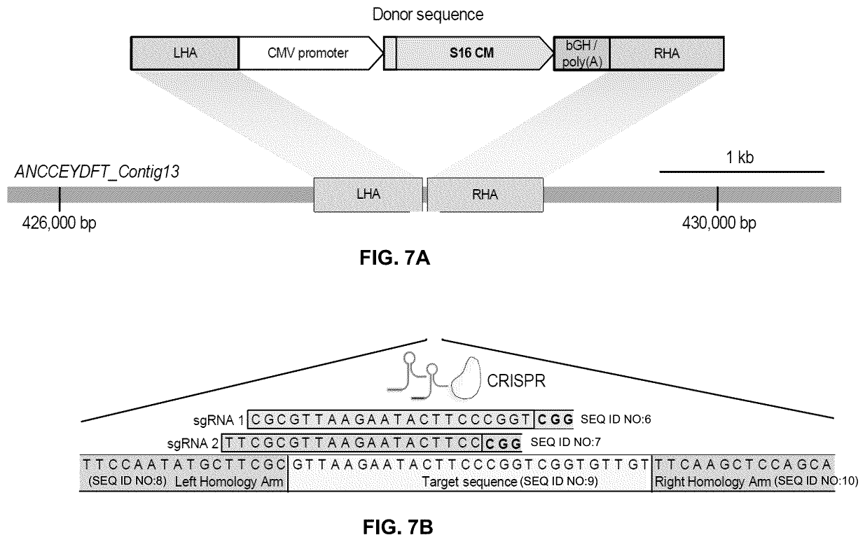

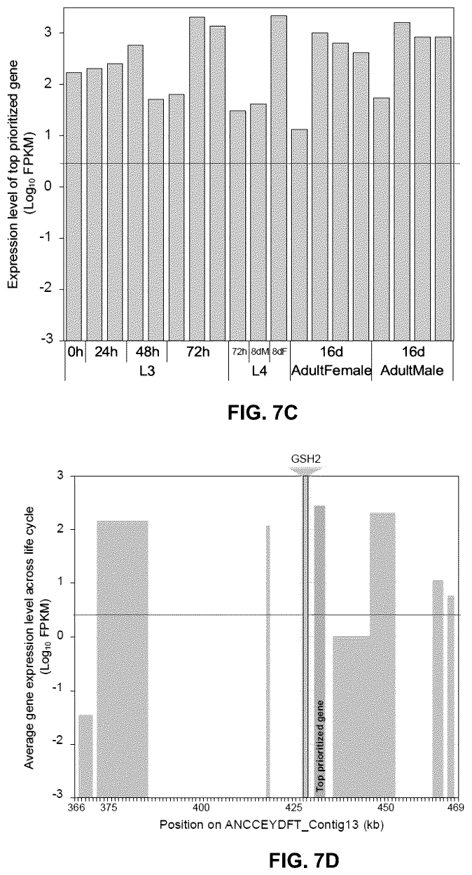

A CRISPR/Cas-9 knock-in system was used for the targeted insertion of the anti-tetrodotoxin (TTX) gene, S16-HuScFv, at the GSH of the human hookworm Ancyclostoma ceylanicum (ANCCEYDFT_Contig13) (FIG. 7A). This gene region has consistent gene level across the A. ceylanicum life cycle (FIG. 7C) compared to other genes in the A. ceylanicum genome (FIG. 7D), making it a good location to knock-in the construct. The construct was engineered by cloning the human single-chain antibody fragment (S16-HuScFv) previously selected from a human ScFv phage display library and shown to neutralize tetrodotoxin into pCDNA3.1(+) backbone with the CMV promoter and BGH terminator. The left homology arm (LHA, SEQ ID NO:8) is situated at the position of two overlapping sgRNAs, sgRNA 1 (SEQ ID NO:6) and sgRNA 2 (SEQ ID NO:7) to allow for knock-in at the target sequence (SEQ ID NO:9) as illustrated schematically at FIG. 7B.

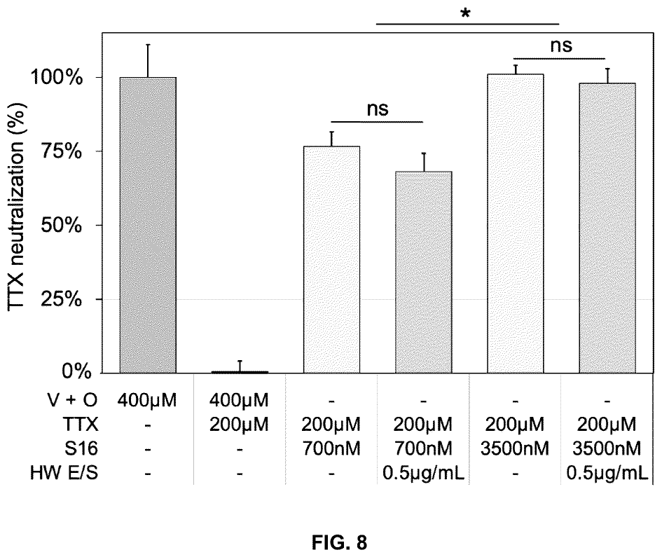

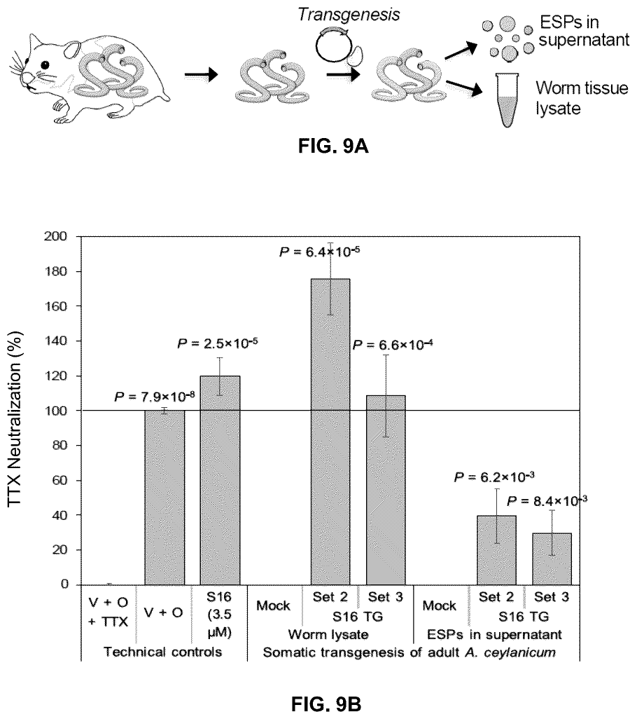

It was then determined that the natural excretory and secretory products (ESP) of hookworms do not interfere with the TTX neutralization potential of S16-HuScFv. Recombinant S16-HuScFv was incubated with 200 nM TTX before in vitro exposure to Neura-2a cells and the percent of TTX neutralized was measured. There was no significant difference in TTX neutralization when hookworm excretory and secretory products were added to the recombinant S16-HuScFv and TTX mixture (FIG. 8). The somatic transgenesis of the S16-HuScFv gene was integrated into hookworms using a programmed CRISPR-Cas9 knock-in donor construct and electroporation. The ability of the hookworm to produce the transgene and neutralize TTX was measured. Neura-2a cells exposed to transgenic hookworm lysates were able to fully neutralize TTX, however, cells only given ESP in supernatant did not perform as well as the positive control (V+O) (FIG. 9B).

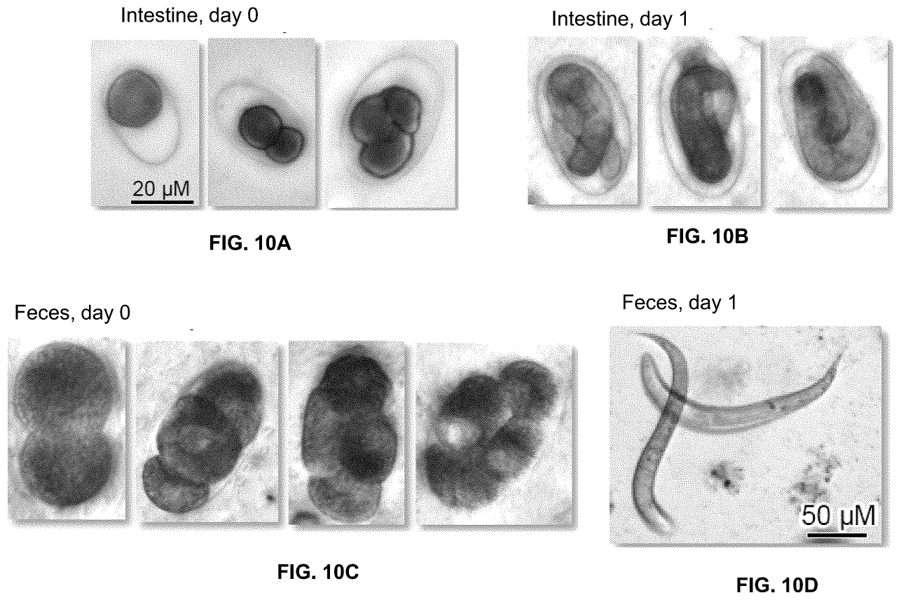

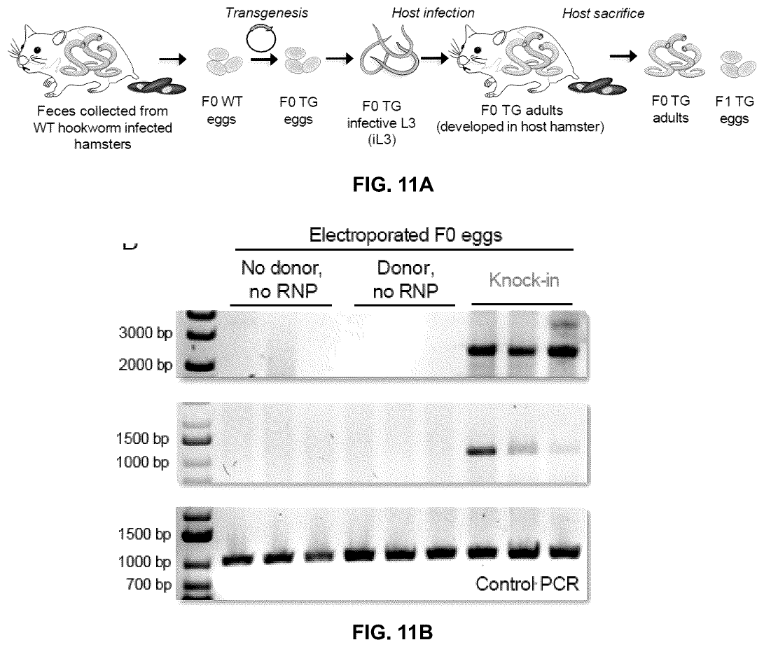

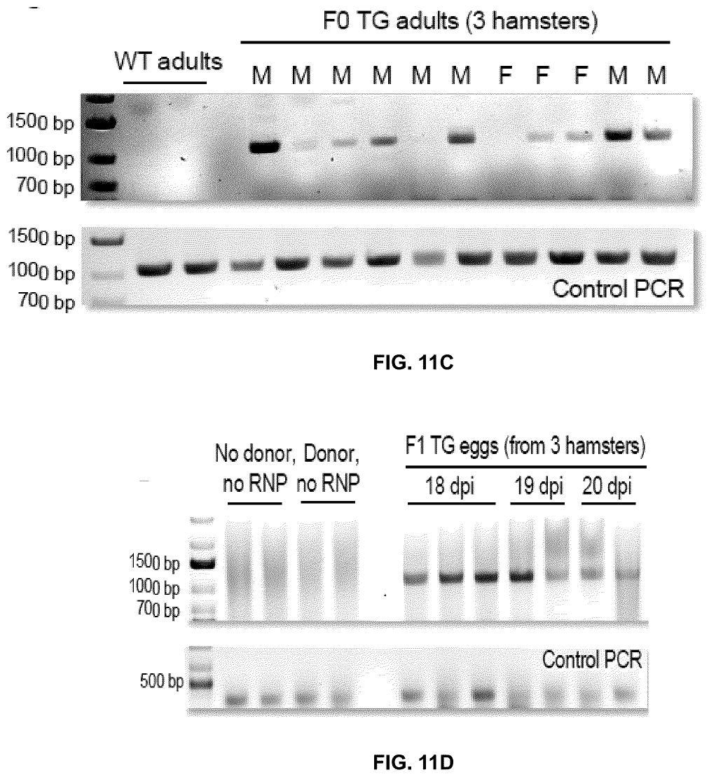

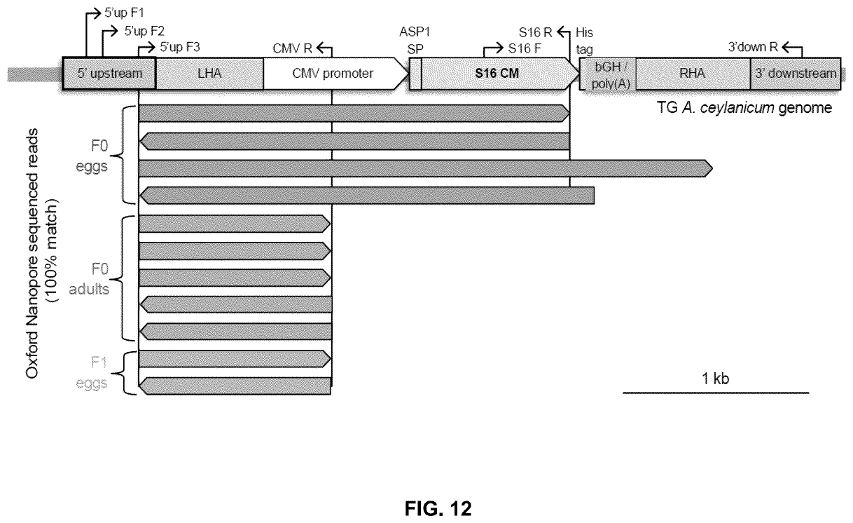

A. ceylanicum was targeted for germline transmission of S16-HuScFv. Immature eggs were extracted from the intestine (FIG. 10A, FIG. 10B) and mature eggs were extracted from hamster fecal samples (FIG. 10C, FIG. 10D) at 20 days post-infection. Immature and mature eggs were observed on day 0 and day 1. To confirm targeted germline knock-in of S16-HuScFv, wild-type hookworm eggs (F0) were collected and transfected with the transgene. F0 larva infected a hamster and developed into adults (F0) and laid F1 eggs (FIG. 11A). F0 eggs (FIG. 11B), F0 adults (FIG. 11C), and F1 eggs (FIG. 11D) were confirmed to have the transgene by PCR amplification. Knock-in was additionally verified using Oxford Nanopore Technology sequencing (FIG. 12).

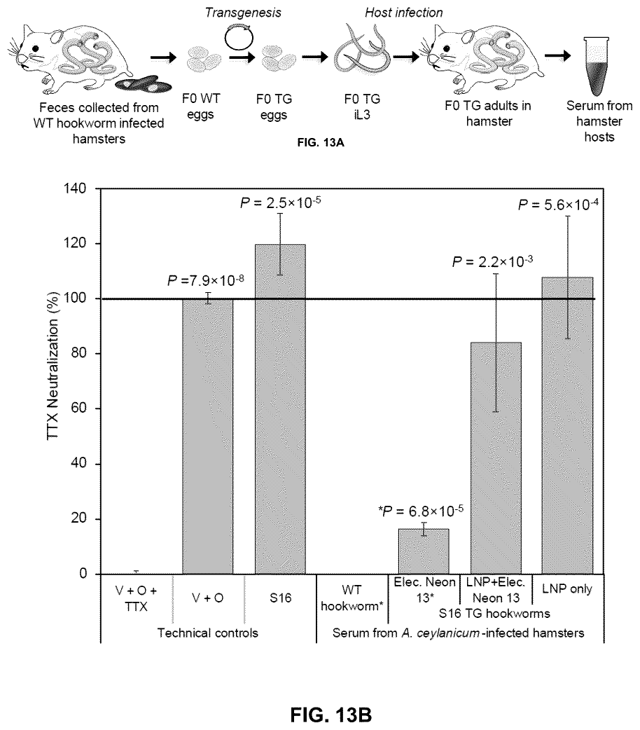

While the S16-HuScFv gene is successfully integrated into A. ceylanicum genome using electroporation, it did not result in robust TTX neutralization (FIG. 9B), thus, the use of lipid nanoparticles (LNP) is also examined for plasmid integration. Wildtype hookworm eggs are collected, transfected, and the resulting larval stage hookworms are used to infect a hamster host. Host hamster serum is then collected for a TTX neutralization assay (FIG. 13A). As previously demonstrated, the serum from hamsters hosting hookworm which were transfected using electroporation, only have a 20% TTX neutralization. When hookworm transfection uses LNP+electroporation or LNP only, the host hamster serum demonstrates TTX neutralization levels similar to the positive control (FIG. 13B). Using this system, the host of transgenic S16-HuScFv hookworms is protected against and can neutralize the neurotoxin tetrodotoxin.

What is claimed is:

1. A composition for the treatment of a disease, condition, or indication in a patient in need, the composition comprising a transgenic hookworm configured to release at least one therapeutic agent, the transgenic hookworm comprising a hookworm genome with a configurable chassis inserted, the configurable chassis comprising:
a. at least one transgenic DNA sequence encoding at least one therapeutic agent, wherein the at least one transgenic DNA sequence encodes the at least one therapeutic agent;
b. at least one promoter DNA sequence operatively linked to the at least one transgenic DNA sequence, the at least one promoter DNA sequence encoding at least one promotor configured to drive expression of the at least one transgenic DNA sequence; and
c. at least one regulatory DNA sequence operatively linked to the at least one transgenic DNA sequence, the at least one regulatory DNA sequence configured to enhance production of the at least one therapeutic agent.

2. The composition of claim 1, wherein the transgenic hookworm is generated by a transgenesis process selected from adult stage random integration transgenesis, egg stage random integration transgenesis, and targeted transgenesis.

3. The composition of claim 1, wherein the at least one promoter is selected from UBI Schisto, EFT3, ACT2, or UBI.

4. The composition of claim 3, wherein the at least one promoter is EFT3.

5. The composition of claim 1, wherein the at least one regulatory DNA sequence comprises at least one signaling peptide DNA sequence encoding at least one signaling peptide selected from Asp2, Asp1, Ap1, 225, and 382, wherein the at least one signaling peptide is configured to promote translocation of the at least one therapeutic agent to a cell membrane of the transgenic hookworm.

6. The composition of claim 5, wherein the at least one signaling peptide is ASP1.

7. The composition of claim 1, wherein the disease, condition, or indication treated by the composition is selected from exposure to a chemical/biological (CB) agent, an inflammatory/autoimmune disease, or a bacterial/viral infection.

8. The composition of claim 1, wherein the transgenic hookworm comprises a species selected from Ancylostoma caninum or Ancylostoma ceylanicum.

9. A method to treat a disease, condition, or indication in a subject, the method comprising:
a. providing a composition comprising a transgenic hookworm configured to release at least one therapeutic agent, the transgenic hookworm comprising a hookworm genome with a configurable chassis inserted, the configurable chassis comprising:
i. at least one transgenic DNA sequence encoding at least one therapeutic agent, wherein the at least one transgenic DNA sequence encodes the at least one therapeutic agent,
ii. at least one promoter DNA sequence operatively linked to the at least one transgenic DNA sequence, the at least one promoter DNA sequence encoding at least one promotor configured to drive expression of the at least one transgenic DNA sequence; and
iii. at least one regulatory DNA sequence operatively linked to the at least one transgenic DNA sequence, the at least one regulatory DNA sequence configured to enhance production of the at least one therapeutic agent; and
b. administering the transgenic hookworm to the gut of the subject.

10. The method of claim 9, wherein the at least one promoter is selected from UBI Schisto, EFT3, ACT2, or UBI.

11. The method of claim 10, wherein the at least one promoter is EFT3.

12. The method of claim 9, wherein the at least one regulatory DNA sequence comprises at least one signaling peptide DNA sequence encoding at least one signaling peptide selected from Asp2, Asp1, Ap1, 225, and 382, wherein the at least one signaling peptide is configured to promote translocation of the at least one therapeutic agent to a cell membrane of the transgenic hookworm.

13. The method of claim 12, wherein the at least one signaling peptide is ASP1.

14. The method of claim 9, wherein the disease, condition or indication treated is exposure to a chemical/biological (CB) agent, an inflammatory/autoimmune disease, or a bacterial/viral infection.

15. The method of claim 9, wherein the transgenic hookworm is administered by oral administration of transgenic hookworm ova, transgenic hookworm larvae, transgenic adult hookworms, and any combination thereof.

16. The method of claim 1, wherein the transgenic hookworm comprises a species selected from Ancylostoma caninum or Ancylostoma ceylanicum.


SEQUENCE LISTING (12 sequences)

SEQ ID NO: 1 - RNA, 12 nt, synthetic construct
  source 1..12: mol_type = other RNA
tactcaggttca

SEQ ID NO: 2 - DNA, 10 bp, synthetic construct
  source 1..10: mol_type = other DNA
aatcaagtca

SEQ ID NO: 3 - DNA, 11 bp, synthetic construct
  source 1..11: mol_type = other DNA
cctgatctaaa

SEQ ID NO: 4 - DNA, 13 bp, synthetic construct
  source 1..13: mol_type = other DNA
agctttcgtctaa

SEQ ID NO: 5 - DNA, 15 bp, synthetic construct
  source 1..15: mol_type = other DNA
aacctggcagttgag

SEQ ID NO: 6 - DNA, 25 bp, synthetic construct
  source 1..25: mol_type = other DNA
cgcgttaagaatacttcccggtcgg

SEQ ID NO: 7 - DNA, 23 bp, synthetic construct
  source 1..23: mol_type = other DNA
ttcgcgttaagaatacttcccgg

SEQ ID NO: 8 - DNA, 16 bp, synthetic construct
  source 1..16: mol_type = other DNA
ttccaatatgcttcgc

SEQ ID NO: 9 - DNA, 28 bp, synthetic construct
  source 1..28: mol_type = other DNA
gttaagaatacttcccggtcggtgttgt

SEQ ID NO: 10 - DNA, 14 bp, synthetic construct
  source 1..14: mol_type = other DNA
ttcaagctccagca

SEQ ID NO: 11 - DNA, 15 bp, synthetic construct
  source 1..15: mol_type = other DNA
ggtaaacgtgtttac

SEQ ID NO: 12 - DNA, 62 bp, synthetic construct
  source 1..62: mol_type = other DNA
ggtaaacgtgtttacgcccgtgttaacgatgggtaaatttgtgtggtgtaaacacgtttacc